(19) United States
(12) Patent Application Publication
(10) Pub. No.: US 2024/0099666 A1
(43) Pub. Date: Mar. 28, 2024

(54) Staging, Paging, and Engagement System for Real-Time Management for Patients at Risk for Cardiogenic Shock

(71) Applicant: Mayo Foundation for Medical Education and Research, Rochester, MN (US)

(72) Inventors: Parag C. Patel, Jacksonville, FL (US); Pablo Moreno Franco, Ponte Vedra Beach, FL (US); Kristine M. Thompson, St. Augustine, FL (US); Rohan M. Goswami, Jacksonville, FL (US); Jacob C. Jentzer, Rochester, MN (US)

(21) Appl. No.: 18/476,099

(22) Filed: Sep. 27, 2023

Related U.S. Application Data
(60) Provisional application No. 63/410,333, filed on Sep. 27, 2022.

(51) Int. Cl. A61B 5/00; G16H 10/65; G16H 50/20; G16H 70/00; G16H 80/00

(52) U.S. Cl. CPC A61B 5/7275; A61B 5/7267; A61B 5/742; G16H 10/65; G16H 50/20; G16H 70/00; G16H 80/00

(57) ABSTRACT

Real-time predictive tools identify patients who are probable to progress to higher stages of shock (e.g., cardiogenic shock and/or non-cardiogenic shock). The tools can be implemented with three modules: a staging module for generating a shock stage based on continuous real-time monitoring of patient health data, a paging module for generating an alert to one or more health care providers, and an engagement module for generating and communicating an updated order set for the patient.

### BACKGROUND

Until recently, the management of cardiogenic shock has been variable with late identification of shock still of concern, limiting the utility of advanced therapies. For this reason, multidisciplinary shock teams have been established with standardized management approaches showing promise in improving outcomes. While these initiatives have focused on improving standard of care by optimizing multidisciplinary management of cardiogenic shock, there are no programs that have leveraged early prospective patient identification using real-time electronic medical record (“EMR”) monitoring integrated with individualized management strategies adapted to the severity of cardiogenic shock.

Several previously described approaches to predict the subsequent development of cardiogenic shock using a one-time static assessment of risk factors, typically at hospital admission, exist. For instance, the Observatoire Regional Breton sur l′Infarctus (“ORBI”) risk score predicts the likelihood of developing in-hospital cardiogenic shock in patients with ST elevation myocardial infarction (“MI”) receiving percutaneous coronary intervention. Biomarkers including natriuretic peptides and ST2 have been shown to be predictive of delayed cardiogenic shock development in ST elevation MI. Integration of biomarkers with clinical features into a risk score has subsequently been utilized as a stratification tool for generalized cardiogenic shock and acute MI cardiogenic shock mortality to allow for prognostication prior to any intervention and or therapies. These predictors primarily have been implemented to determine utility or futility of interventions based on clinical criteria, but are not able to provide real-time predictive tools to identify which patients will likely progress to higher stages of shock. Moreover, these previous tools do not provide any dynamic risk stratification, representing a substantial limitation to their practical usefulness.

Cardiogenic shock risk scores proposed to date have been limited on their temporal assessment of patient risk, as they are based on admission data and do not take into account the fluid nature of cardiogenic shock, which changes during the entire period of the hospitalization. It has been proposed that patient health records can be leveraged by describing the development of a digital phenotype using electronic health record (“EHR”) data to stratify patients by risk of decompensation using the following variables: hypotension, organ dysfunction, hypoperfusion, vasoactive use, respiratory decline and respiratory intervention. These data emphasize that patients can develop increasing and decreasing risks for cardiogenic shock at different time points during their hospitalization.

### SUMMARY OF THE DISCLOSURE

The present disclosure addresses the aforementioned drawbacks by providing a method for generating an order set for a patient based a shock stage. The method includes receiving patient health data with a computer system, where the patient health data are associated with a patient and are continuously received in a real-time manner. A staging algorithm is also accessed with the computer system, where the staging algorithm is configured to generate shock stage classification data from patient health data. The patient health data are input to the staging algorithm using the computer system as the patient health data are continuously received by the computer system in real-time, thereby generating shock stage classification data for the patient as an output. An alert is generated with the computer system when the shock stage classification data for the patient indicate a change in a shock stage. An order set is generated by the computer system, in response to the alert, based on the shock stage classification data for the patient. The order set is stored in an EMR for the patient using the computer system. Other embodiments of this aspect include corresponding systems (e.g., computer systems), programs, algorithms, and/or modules, each configured to perform the steps of the methods.

It is another aspect of the present disclosure to provide a non-transitory computer-readable media having stored thereon instructions that when executed by a processor cause the processor to perform a method that includes retrieving patient health data from a data storage in real-time, where the patient health data are associated with a patient; accessing a machine learning model trained on training data to generate shock stage classification data from patient health data; generating shock stage classification data for the patient in real-time by inputting the patient health data to the machine learning model as the patient health data are continuously retrieved from the data storage; and storing the shock stage classification data using the processor.

It is yet another aspect of the present disclosure to provide a system for shock staging. The system includes a staging module to generate shock stage classification data by receiving patient health data in real-time, and applying the patient health data to a staging model that classifies the patient health data as including features that are correlated with a particular shock stage classification. The system also includes a paging module to generate an alert in response to the shock stage classification data indicating a change in a shock stage and an engagement module to generate an updated order set for the patient in response to the shock stage classification data indicating the change in the shock stage.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIG. 1 is a workflow overview of an example stage, page, and engage platform in accordance with some embodiments described in the present disclosure.

FIG. 2 is a block diagram of an example system implementing a stage, page, and engage system.

FIG. 3 is an example shock stage scoring system, in which a unique clinical score is derived and automatically assigned to a corresponding SCAI shock stage based on six clinical variables.

FIG. 4 illustrates an example cardiogenic shock Care Path staging system demonstrating escalation and de-escalation pathways for all The Society for Cardiovascular Angiography and Interventions (“SCAI”) stages. On the left, SCAI cardiogenic shock stages are displayed as an alert in real-time. The arrows within the Care Path demonstrate all the possible escalation and de-escalation paths for the patient. Stages and arrows highlighted in blue represent the different paths the patient entered during the hospital course and the stage highlighted in orange represents the current stage.

FIG. 5 illustrates an example tier alert system. SCAI cardiogenic shock stages C-E were assigned a corresponding Tier from 1-3. Based on the Tier activated, a pre-determined multidisciplinary team is alerted by page. For Tier 2 or greater alerts, immediate communication via a virtual videoconference room allows for rapid management decisions.

FIG. 6 is a flowchart illustrating an example method for implementing a stage, page, and engage platform.

FIG. 7 illustrates a complex real-time pathway-based relation tree setting forth an example workflow for implementing shock staging, paging, and engaging for multiple different shock types.

FIG. 8 is a block diagram of an example system for implementing a stage, page, and engage platform.

FIG. 9 is a block diagram of example components that can implement the system of FIG. 8.

### DETAILED DESCRIPTION

Described here systems and methods for real-time predictive tools to identify patients who are probable to progress to higher stages of cardiogenic shock or other non-cardiogenic shock types or mixed shock types (e.g., mixtures of cardiogenic and non-cardiogenic shock types, mixtures of different non-cardiogenic shock types). As a non-limiting example, non-cardiogenic shock can include other types of circulatory shock including disruptive shock (e.g., septic shock, anaphylactic shock, neurogenic shock), hypovolemic shock (e.g., hemorrhagic shock), and/or obstructive shock. Noncardiogenic shock may also include other non-circulatory shock types, including insulin shock. The disclosed systems and methods are also capable of risk stratifying other disease processes, conditions, or states other than shock types, such as pulmonary embolism, or the like.

Advantageously, the disclosed systems and methods are capable of providing dynamic risk stratification. In some embodiments, the systems and methods can be implemented with three modules: a staging module, a paging module, and an engagement module. As such, in some embodiments the disclosed systems may be referred to as a stage, page, and engage (“SPE”) system.

Nationally, there are three primary challenges that contribute to high mortality in patients with cardiogenic shock: late identification of patients in cardiogenic shock, suboptimal communication by multidisciplinary teams for cardiogenic shock management, and heterogeneous approaches in hemodynamic management of cardiogenic shock. The systems and methods described in the present disclosure address these problems by providing a real-time continuous EMR-based (or EHR-based) classification of patients into shock categories aimed at earlier identification of patients in cardiogenic shock, or non-cardiogenic shock, so multidisciplinary teams can be alerted and patient management engaged earlier than possible before. Advantageously, the systems and methods described in the present disclosure provide a medical tool can provide for a significant improvement on patient survival rates leading to explant. By way of example, implementing the systems and methods described in the present disclosure in a clinical setting improved survival rates from 35% to 89-94%. These statistics underscore the efficacy of the disclosed systems and methods in significantly improving patient outcomes, thereby provided enhanced patient care and clinical practice.

In an example embodiment, the staging module of the SPE system provides a prospective continuous real-time electronic medical record (“EMR”) and/or electronic health record (“EHR”) care path that continuously categorizes patients into cardiogenic shock stages, or non-cardiogenic shock stages. As a non-limiting example, the patients may be categorized into Society for Cardiovascular Angiography and Intervention (“SCAI”) cardiogenic shock stages. SCAI cardiogenic shock stages provide a classification system to categorize the severity spectrum of cardiogenic shock into escalating stages from Stage A (those at risk of developing shock) through Stage E (those in extremis with impending circulatory collapse). These categories can be displayed to a user (e.g., a clinician) via a client device or in the patient's EMR and/or EHR, thereby allowing for clinicians to risk stratify patients at risk for cardiogenic shock. Additionally or alternatively, the patients may be categorized by including other high risk and low risk stages to identify patients at risk for deterioration such that closer monitoring of those patients can be completed, or such that management escalation can be considered for higher risk patients. Thus, in some implementations the staging module may generate predictive scores that indicate a likelihood of deterioration for a particular patient. In these instances, the staging module may provide a classification of the patient (e.g., by stratifying patients into categories, such as those described above), a prediction of deterioration of the patient, or both.

The staging module can then be integrated with the paging module, which may include an automated EMR-based and/or EHR-based paging alert system and/or tier alert system that notifies clinicians of a patient's declining state so that tailored therapies can be applied based on shock severity. Thus, in some implementations, the staging module generates classified feature data that include a cardiogenic shock stage (e.g., an SCAI stage), a cardiogenic shock staging score value, non-cardiogenic shock classifications and/or score values, or the like, which are then evaluated with the paging module to assess whether a patient alert and/or tier alert should be generated based on the output of the staging module.

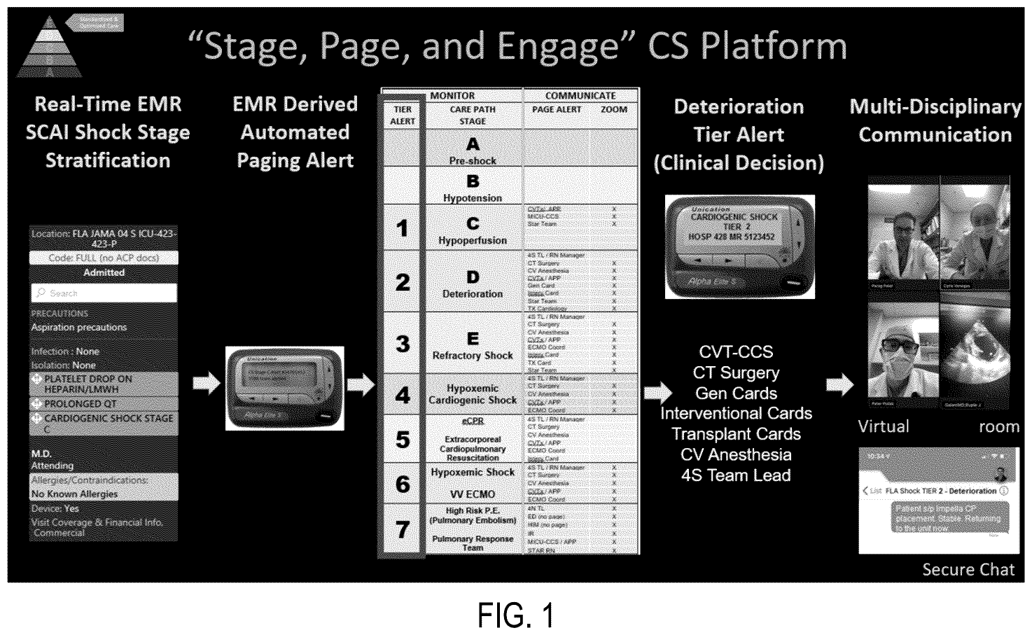

FIG. 1 depicts an example workflow that can be implemented with an SPE system according to some embodiments described in the present disclosure. In this example, an overview of a stage, page, and engage cardiogenic shock platform is illustrated. As described above, in other instances the stage, page, and engage platform may be additionally or alternatively implemented for non-cardiogenic shock types. In the illustrated example, EMR-based and/or EHR-based real-time stratification of SCAI categories triggers an EMR and/or EHR derived automated paging alert for higher SCAI stages. The primary team receiving this alert will initiate an order set that is tailored to the SCAI cardiogenic shock stage. For instance, stage B order sets provide increased monitoring of vitals and labs, while stage C order sets initiate an evaluation for shock differentiation. The primary team can escalate care in deteriorating patients (e.g., SCAI stages C-E) using a tiered alert system that pages a multidisciplinary group unique to each tier, which in some implementations immediately joins a virtual videoconference room where the group can discuss the patient state to determine a management plan. Subsequently, patient information and updates can be rapidly disseminated to the multidisciplinary group using a HIPAA compliant messaging platform.

In some embodiments, patient entry into the care path that involves SCAI stage stratification (e.g., the staging module of the SPE system) can be automated for all patients on the cardiology, ICU, and CT surgery services and/or for selected patients on the general medicine services, based on admission diagnosis. An automated paging alert activation system can notify the health care team of increasing shock stages and order sets can determined based on shock stage. As a non-limiting example, these order sets can increase frequency of hemodynamic and laboratory monitoring for SCAI stage B, rapidly implement studies to assist with shock differentiation for SCAI stage C, and have the potential to activate a tier alert for consideration of advanced therapies including temporary mechanical support and ICU care for SCAI stages D and E.

Similar care path platforms with a scoring algorithm can be readily adapted and applied to other shock types (e.g., non-cardiogenic shock) and/or disease processes, including advanced pulmonary embolism, to stratify risk and tailor therapies. These platforms can be highly beneficial to users and applications aiming to utilize advanced therapies, such as mechanical circulatory support, earlier in a disease process. Hospital-wide platforms that integrate continuous EMR-based and/or EHR-based monitoring and prediction with a multidisciplinary alert system have the potential to favorably affect outcomes within this patient population.

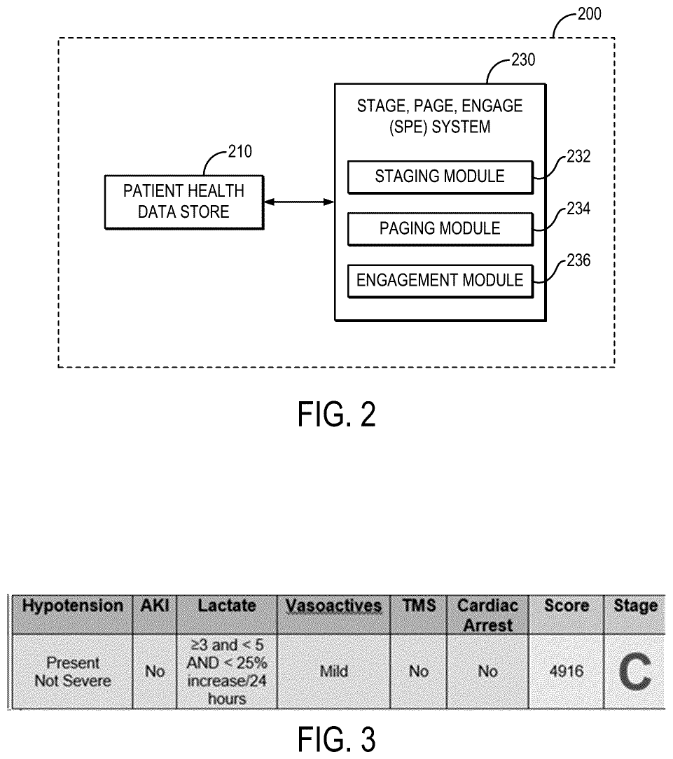

FIG. 2 illustrates an example system 200 that can implement the stage, page, and engage platforms described in the present disclosure. The system 200 generally includes a patient health data store 210 that is in communication with an SPE system 230. As described above, the SPE system 230 includes a staging module 232, a paging module 234, and an engagement module 236. In some implementations, the SPE system 230 can be implemented as part of an EMR system (e.g., part of a hospital-wide EMR system) and/or EHR system.

The patient health data store 210 can include one or more databases or other data storage devices that store patient health data. The patient health data may include data stored in, retrieved from, extracted from, or otherwise derived from the patient's electronic medical record (“EMR”) and/or electronic health record (“EHR”). The patient health data can include unstructured text, questionnaire response data, clinical laboratory data, histopathology data, genetic sequencing, medical imaging, and other such clinical data types. Examples of clinical laboratory data and/or histopathology data can include genetic testing and laboratory information, such as performance scores, lab tests, pathology results, prognostic indicators, date of genetic testing, testing method used, and so on.

In some instances, the patient health data can include one or more types of omics data, such as genomics data, proteomics data, transcriptomics data, epigenomics data, metabolomics data, microbiomics data, and other multiomics data types. The patient health data can additionally or alternatively include patient geographic data, demographic data, and the like. In some instances, the patient health data can include information pertaining to diagnoses, responses to treatment regimens, genetic profiles, clinical and phenotypic characteristics, and/or other medical, geographic, demographic, clinical, molecular, or genetic features of the patient.

Features derived from structured, curated, and/or EMR or EHR data may include clinical features such as diagnosis, symptoms, therapies, outcomes, patient demographics such as patient name; date of birth; gender; ethnicity; diagnosis dates for cancer, illness, disease, or other physical or mental conditions; personal medical history; family medical history; clinical diagnoses, such as date of initial diagnosis, date of metastatic diagnosis, cancer staging, tumor characterization, and tissue of origin. Additionally, the patient health data may also include features such as treatments and outcomes, such as line of therapy, therapy groups, clinical trials, medications prescribed or taken, surgeries, radiotherapy, imaging, adverse effects, and associated outcomes. As a non-limiting example, the patient health data may include baseline demographics, past medical history, ICD-10 discharge diagnoses, 30-day all-cause mortality, mean intubation days, mean ICU data, need for renal replacement therapy (“RRT”), and/or need for temporary mechanical support (“TMS”).

Patient health data can include a set of clinical features associated with information derived from clinical records of a patient, which can include records from family members of the patient. These clinical features and data may be abstracted from unstructured clinical documents, EMR, EHR, or other sources of patient history. Such data may include patient symptoms, diagnosis, treatments, medications, therapies, responses to treatments, laboratory testing results, medical history, geographic locations of each, demographics, or other features of the patient which may be found in the patient's EMR and/or EHR.

In some instances, patient health data can include medical imaging data, which may include images of the patient obtained with one or more different medical imaging modalities, including magnetic resonance imaging (“MRI”), computed tomography (“CT”), x-ray imaging, positron emission tomography (“PET”), ultrasound, and so on. The medical imaging data may also include parameters or features computed or derived from such images. Medical imaging data may also include digital pathology images, such as hematoxylin and eosin (“H&E”) stain slides, immunohistochemistry (“IHC”) slides, and the like. The medical imaging data may also include data and/or information from pathology and radiology reports, which may be ordered by a clinician during the course of diagnosis and treatment of various illnesses and diseases.

As a non-limiting example, epigenomics data may include data associated with information derived from DNA modifications that are not changes to the DNA sequence and regulate the gene expression. These modifications can be a result of environmental factors based on what the patient may breathe, eat, or drink. These features may include DNA methylation, histone modification, or other factors which deactivate a gene or cause alterations to gene function without altering the sequence of nucleotides in the gene.

Microbiomics data may include, for example, data derived from the viruses and bacteria of a patient. These features may include viral infections which may affect treatment and diagnosis of certain illnesses as well as the bacteria present in the patient's gastrointestinal tract which may affect the efficacy of medicines ingested by the patient.

Proteomics data may include data associated with information derived from the proteins produced in the patient. These features may include protein composition, structure, and activity; when and where proteins are expressed; rates of protein production, degradation, and steady-state abundance; how proteins are modified, for example, post-translational modifications such as phosphorylation; the movement of proteins between subcellular compartments; the involvement of proteins in metabolic pathways; how proteins interact with one another; or modifications to the protein after translation from the RNA such as phosphorylation, ubiquitination, methylation, acetylation, glycosylation, oxidation, or nitrosylation.

The staging module 232 is configured to prospectively assign shock stages (e.g., SCAI shock stages) in real-time using patient health data received from the patient health data store 210. The patient shock stages may be determined by inputting the patient health data to one or more staging algorithms, programs, or models implemented by the staging module 232. In some examples, the algorithms, programs, or models may include deep learning, machine learning, or other artificial intelligence (“AI”) based algorithms, programs, or models.

As one non-limiting example, the staging module 232 may implement a staging algorithm, program, or model that has been trained on training data using supervised learning. As an example, supervised learning involves presenting a computer system with example inputs and their actual outputs (e.g., categorizations). In these instances, the staging algorithm or model is configured to learn a general rule or model that maps the inputs to the outputs based on the provided example input—output pairs. For instance, the staging algorithm or model may be trained to receive patient health data as input data in order to generate shock stage classification data as output data, where the shock stage classification data are indicative of a predicted or otherwise estimated shock stage for the patient based on the real-time interpretation of their patient health data (e.g., the shock stage classification data indicate that the patient health data include features that are correlated with a particular shock stage classification). The training data may, therefore, include patient health data paired with clinical outcomes (e.g., clinically determined cardiogenic shock stages, clinically determined non-cardiogenic shock stages) collected from a population or group of patients. In some embodiments, the training data may include patient health data that have been labeled (e.g., labeled as containing patterns, features, or characteristics indicative of different cardiogenic and/or non-cardiogenic shock stages; and the like).

A staging algorithm or model may thus be trained on the training data. In general, the staging algorithm or model can be trained by optimizing parameters (e.g., weights, biases, other model parameters) based on minimizing a loss function (e.g., a mean squared error loss function), or the like. As a non-limiting example, training a machine learning model may include initializing the model, such as by computing, estimating, or otherwise selecting initial model parameters (e.g., weights, biases, or other model parameters).

During training, the initial machine learning model receives the inputs for a training example and generates an output (e.g., using the bias for each node in a neural network and the connections between each node and the corresponding weights). For instance, training data can be input to the initialized machine learning model, generating output as shock stage classification data. The generated output is then compared with the actual output of the training example in order to evaluate the quality of the shock stage classification data. For instance, the shock stage classification data can be passed to a loss function to compute an error. The current machine learning model can then be updated based on the calculated error (e.g., using backpropagation methods based on the calculated error). For instance, the current machine learning model can be updated by updating the model parameters (e.g., weights, biases, or other model parameters) in order to minimize the loss according to the loss function.

The training continues until a training condition is met. The training condition may correspond to, for example, a predetermined number of training examples being used, a minimum accuracy threshold being reached during training and validation, a predetermined number of validation iterations being completed, and the like. When the training condition has been met (e.g., by determining whether an error threshold or other stopping criterion has been satisfied), the current machine learning model and its associated model parameters represent the trained machine learning model. The training processes may include, for example, gradient descent, Newton's method, conjugate gradient, quasi-Newton, Levenberg-Marquardt, among others

As noted above, inputting the patient health data to the staging algorithm generates output data as shock stage classification data indicating a predicted or otherwise estimated shock stage for the patient based on the real-time interpretation of their patient health data.

For example, an EMR-based and/or EHR-based prospective continuous real-time staging algorithm can be implemented using a care path workflow that continuously stratifies at-risk patients into different shock stages (e.g., SCAI shock stages for cardiogenic shock, other risk levels or stages for non-cardiogenic shock types). Scores assigned to various categorical variables (e.g., six categorical variables in one example) can be utilized to develop unique clinical states of cardiogenic and/or non-cardiogenic shock. As a non-limiting example, the variables utilized can include measures of hypotension, lactate, vasopressor use, renal function, temporary mechanical support, and cardiac arrest. In this example, each of the unique clinical states can be assigned a SCAI classification a priori based on perceived risk as determined by a multidisciplinary group consensus including cardiology, intensivists, transplant cardiology, and interventional cardiology. FIG. 3 illustrates an example scoring system that can be used to stratify patients into SCAI cardiogenic shock stages.

Patients enrolled in the care path can be monitored and staged in real-time by the staging module 232 of the SPE system 230 based on the staging algorithm during each data entry point. A clinical decision support alert with the patient's unique clinical score and/or shock stage can be displayed prominently in the patient chart. The staging algorithm escalates the shock stage at any point of a patient's hospital course based on flowsheet entry, medication administration, device implantation, lab values, or other patient health data received from the patient health data store 210 and can de-escalate the shock stage after a preset time period (e.g., six hours of entering a higher stage) if de-escalation criteria are met. In this way, the SPE system 230 is capable of continuously staging and stratifying patients based on shock severity.

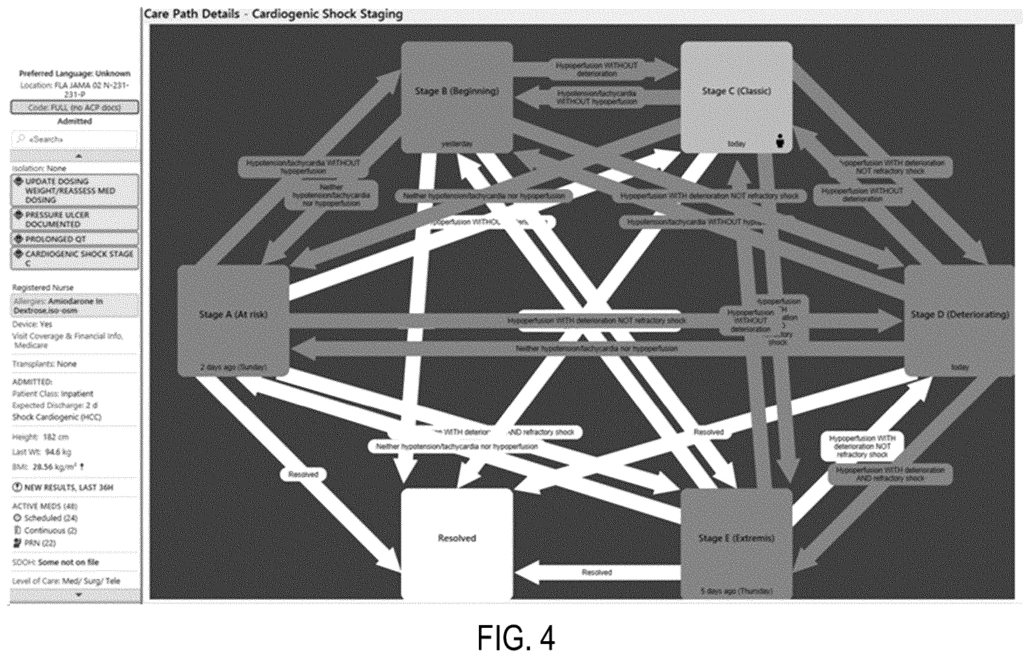

FIG. 4 illustrates an example care pathway of an example patient and their SCAI staging algorithms. This figure depicts the dynamic and continuously changing states that a patient may have during the course of his/her illness. On the left, in the display, cardiogenic shock SCAI categories are exhibited in real time. The arrows within the care path demonstrate all the possible escalation and de-escalation paths for patients. Stages and arrows highlighted in blue represent the different paths the patient entered during the hospital course and the stage highlighted in orange represents the current stage.

The paging module 234 is configured to generate one or more alerts based on monitoring the shock stage of the patient as classified by the staging module 232. In this way, the paging module 234 can receive the shock stage classification data output from the staging module 232 and, based on an analysis of those data, can generate a paging alert and/or tiered alert.

Advantageously, the paging module 234 addressed suboptimal communication during previous approaches for cardiogenic and/or non-cardiogenic shock management by providing a streamlined process of patient throughput to optimize therapy selection and timing of delivery. After reviewing the potential entry points of patients within a cardiogenic and/or non-cardiogenic shock system, the paging module 234 can identify one or more members of a multidisciplinary team who should be alerted based on the shock stage classification data for the patient. For instance, the paging module 234 can be configured to retrieve one or more multidisciplinary team member names or IDs from a list of such members based on the SCAI shock stages indicated by the shock stage classification data. As an example, a tier alert system can be applied where, depending on the tier that is activated, a multidisciplinary team is alerted in order to optimize patient care in higher SCAI shock stages.

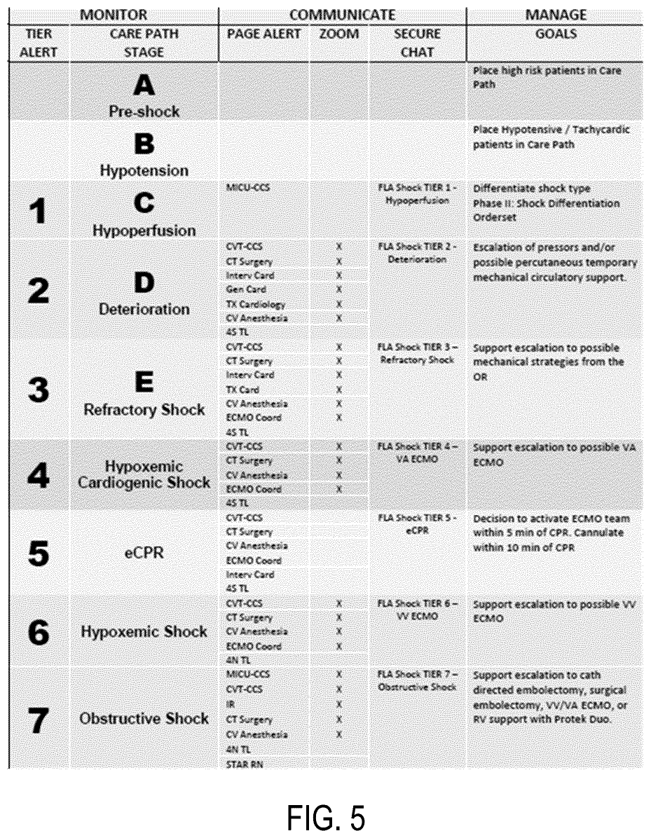

FIG. 5 illustrates an example of SCAI shock and corresponding alert tiers. Stages C-E were assigned a corresponding Tier from 1-3. Based on the tier activated, a pre-determined multidisciplinary team is alerted by page. For Tier 2 or greater alerts, immediate communication via a virtual videoconference room allows for rapid management decisions.

As an example, when the tiered alert system is activated for Tier 2 or greater alerts, an alert (e.g., a page, a text message, a secure message) can be sent to invite a multidisciplinary team to meet immediately in a virtual videoconference room where they can collectively review patient information and images to allow for immediate decision-making regarding therapies for advanced patient care. Depending on the severity of illness, as determined by the multidisciplinary team, the patient may progress to receive additional monitoring, vasopressors, inotropes, or TMS. Using a HIPAA-compliant texting or other messaging platform, (e.g., Epic® Secure Chat) updates regarding patient care may be disseminated to the group. To optimize communication for the sickest patients, emergency tiers can be expanded to include patients who needed VA- or VV-ECMO for any reason and patients with obstructive shock.

The engagement module 236 is configured to generate and communicate an order set for the patient based on user input received following the multidisciplinary team meeting in response to the tier alert system of the paging module 234. In a non-limiting example, the following process was implemented: the cardiovascular-thoracic-transplant ICU was assigned as the primary team for all patients with cardiogenic shock, an integrated medical practice structure was formed within critical care to allow for co-management of complicated patients while optimizing the expertise of each group, and order sets that were tied into the shock best practice advisory for varying levels of shock were generated in order to provide conformity regarding monitoring, labs, and management of patients.

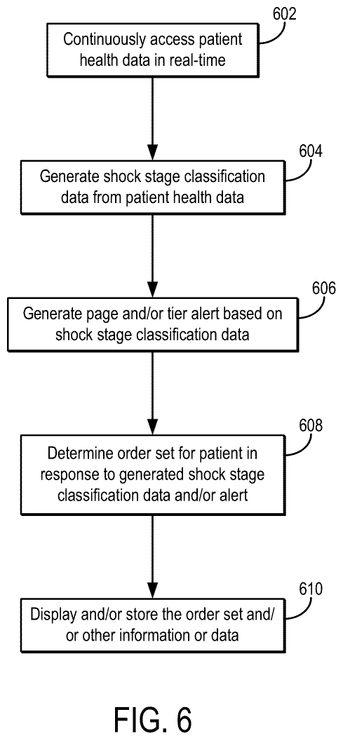

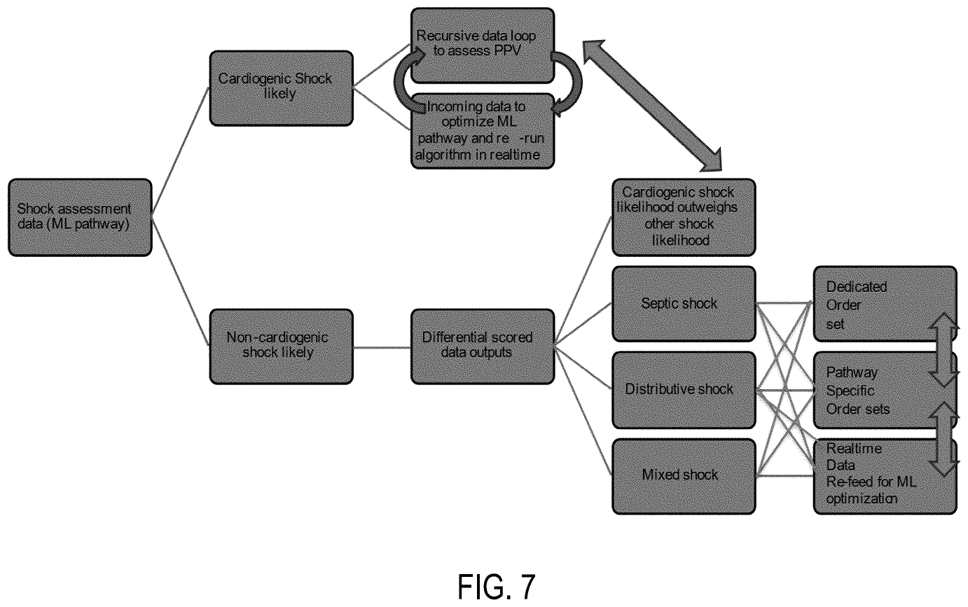

Referring now to FIG. 6, a flowchart is illustrated as setting forth the steps of an example method for implementing a stage, page, and engage platform to continuously monitor and risk stratify a patient.

The method includes receiving or otherwise accessing patient health data with a computer system, as indicated at step 602. Receiving or otherwise accessing the patient health data may include retrieving such data from a memory or other suitable data storage device or medium, such as a patient health data store as described above. Additionally or alternatively, receiving or otherwise accessing the patient health data can include acquiring patient health data from a patient and recording the patient health data with the computer system, or otherwise transferring the patient health data to the computer system.

As described above, the patient health data can be received or otherwise accessed continuous and in real-time while the patient is being monitored in the clinical setting. For instance, the computer system may receive real-time updates of patient health data as there are acquired and sent to, or otherwise accessed by, the computer system. The patient health data may include, for example, measures of hypotension, lactate, vasopressor use, renal function, temporary mechanical support, and cardiac arrest, among other data types described above in more detail.

The patient health data are input to a staging algorithm implemented by the computer system, generating output data as shock stage classification data, as indicated at step 604. For example, the shock stage classification data may include an SCAI shock stage classification (e.g., Stage A, Stage B, Stage C, Stage D, Stage E). Additionally or alternatively, the shock stage classification data may include a shock stage score value, which may include a numerical score value generated from the patient health data. The shock stage score value may be converted to or otherwise correlated with an SCAI shock stage value. As another example, the shock stage classification data may indicate the probability for a particular classification (i.e., the probability that patient is in SCAI Stage A, Stage B, etc.), and/or the probability or risk for escalation or de-escalation from one SCAI shock stage to another.

The shock stage classification data may also include shock stage classifications, shock stage score values, probabilities for a particular classification, and/or probability or risk for escalation or de-escalation from one shock stage to another for non-cardiogenic shock types or other disease processes, conditions, or states other than shock types, such as pulmonary embolism, or the like. As an example, non-cardiogenic shock may include other types of circulatory shock including disruptive shock (e.g., septic shock, anaphylactic shock, neurogenic shock), hypovolemic shock (e.g., hemorrhagic shock), and/or obstructive shock. Noncardiogenic shock may also include other non-circulatory shock types, including insulin shock. The shock stage classification data may also be indicative of mixed shock types (e.g., mixtures of cardiogenic and non-cardiogenic shock types, mixtures of different non-cardiogenic shock types).

Based on the shock stage classification data, the computer system generates an alert when certain criteria are met, as indicated at step 606. For instance, if a patient's shock stage changes from one stage to the next (e.g., SCAI shock stage escalates from Stage B to Stage C), then an alert can be generated by the computer system and transmitted or otherwise communicated to one or more users in a multidisciplinary group of clinical team members. As described above, the alert may include a page, a text message, an email, or other electronic message.

In response to the alert, an order set is determined or otherwise selected for the patient based on the shock stage classification data, as indicated at step 608. In general, an order set includes one or more orderables (e.g., a health care provider's instructions or directives to other health care workers regarding the treatment of the patient). The order set may include a prescription for medication, orders for therapies to be delivered, orders for medical imaging, orders for laboratory testing, and so on.

In some implementations, the health care team can select an order set based on the shock stage classification data, the patient data, and/or other information or data. In some other implementations, an order set can be selected or otherwise determined by the computer system in response to the shock stage classification data and/or patient health data for the patient. In these instances, the order set can be presented to a clinician and/or health care team for review before being accepted and prescribed by the clinician and/or health care team.

As an example, when a patient met SCAI Stage B or higher, an automated paging alert activation can be generated to notify the health care team of increasing shock stages. Based on the page, or other message, the team can activate an order set based on the SCAI stage aimed at 1) increasing frequency of hemodynamic and laboratory monitoring (SCAI B), 2) rapidly implementing studies to assist with shock differentiation (SCAI C), and 3) activating a tier alert for consideration of advanced therapies including TMS and ICU care (SCAI D-E). The continuous SCAI staging is designed to have increased sensitivity of monitoring for Stages B-C to better identify progression in shock stages and specificity.

The selected order set can then be stores in the patient's EMR and/or EHR where it can be acted upon by the health care team, as indicated at step 610.

In an example study, outcome and quality metrics were reviewed among three cohorts. A Historical Controls Cohort (n=529) was retrospectively determined to have CS by ICD-10 diagnosis of shock combined with a cardiac event code, but were not stratified by SCAI shock stage. The Optimization Cohort (n=187) was defined as patients manually enrolled into a Care Path for monitoring and were continuously risk-stratified into SCAI CS stages based on EMR scoring (e.g., via the staging module of the SPE system). Clinicians had the option to manually initiate an emergency Tier Alert, virtual Zoom® Room, and Epic® Secure Chat, when necessary.

All data were extracted from the EMR (Epic®; Verona, WI). Data for the Historical Controls included baseline demographics, past medical history, ICD-10 discharge diagnoses, and 30-day all-cause mortality. Data for the Optimization Cohort included baseline characteristics, past medical history, ICD-10 discharge diagnoses, lab values, and outcomes, including 30-day all-cause mortality, mean intubation days, mean ICU days, need for renal replacement therapy (RRT), and need for TMS. Laboratory values closest to the period of the first transition to the highest documented SCAI CS stage were utilized in the analysis. Utilization of the Tier Alert system was obtained through a database maintained by the hospital communications department. Medical records were retrospectively reviewed to document interventions and survival to discharge of patients requiring a Tier Alert for patient management.

The study population included 529 Historical Controls and 187 Optimization Cohort patients who were manually enrolled in the cardiogenic shock (“CS”) Care Path. Compared to Historical Controls, Optimization Cohort patients were sicker with greater comorbidities.

The maximal SCAI stage was A in five patients, B in 38 patients, C in 19 patients, D in 32 patients, and E in 93 patients. There were no significant differences in baseline demographics, past medical history, and ICD-10 diagnoses based on SCAI stages. Patients with higher shock stages had greater lactic acid levels (p<0.01) and were more commonly anemic (p<0.01). Higher SCAI stages were associated with longer intubation days (p=0.017) and need for renal replacement therapies (pp=0.013). A significant trend towards greater 30-day mortality was demonstrated in higher SCAI stages, supporting this model of stratification with 3%, 16%, 9%, and 25% mortality for stages B, C, D, and E, respectively (p=0.026). Temporary mechanical circulatory support was utilized in 26% of patients in SCAI Stage E. In-hospital mortality in patients requiring TMS improved from 64% to 21% in Optimization Cohort patients requiring TMS (p<0.01).

Eight (23%) alerts were for transfers and 26 (76%) were for inpatients. Of the inpatients requiring a Tier Alert, 19 (73%) required an immediate intervention. Sixteen (61%) of these inpatients required TMS including 13 ECMOs, two Impellas and one IABP. Eight Tier Alert patients (31%) died prior to discharge. TMS was avoided appropriately in two patients due to futility of care in the setting of advanced comorbidities, which contributed to a portion of these deaths.

The systems and methods described in the present disclosure provide a platform of prospectively integrating real-time, continuous patient monitoring to risk-stratify cardiogenic shock patients into SCAI stages in conjunction with an integrated multidisciplinary alert and communication system as part of a comprehensive cardiogenic shock care pathway.

In the example study described above, short-term mortality was higher in patients with higher SCAI stages with more use of renal replacement therapy and longer intubation days. Mean ICU LOS was significantly greater in patients with SCAI Stage E compared to SCAI Stage B-D. A significant reduction in in-hospital mortality for patients requiring TMS was demonstrated (21% vs. 64% at baseline). This beneficial result was achieved by using the coordinated, multidisciplinary care team with early shock identification, rapid assessment, and tailored therapy provided by the SPE system. Additionally, the ability to quickly develop care plans in real-time to provide an efficient, comprehensive cardiogenic shock care plan will reduce not only mortality, but also patient and hospital costs.

The SPE system described in the present disclosure demonstrates the feasibility of continuously monitoring patients throughout their hospitalization using the EMR and/or EHR and integrating it with a system that optimizes multidisciplinary communication and converged management. Development of a tailored platform implements a multidisciplinary approach that identifies strengths and weaknesses of an individual program and a methodical approach to quality initiative development.

To optimize cardiogenic and/or non-cardiogenic shock detection and management, real-time patient stratification into impending, established, and terminal shock can be implemented and joined with tailored therapies based on the level of shock. Given the heterogeneous nature of shock and cardiogenic shock, phenotyping using the SCAI stages or other shock stages or risk levels as a platform is an advantageous option.

Cardiogenic and/or non-cardiogenic shock risk scores proposed to date have been limited on their temporal assessment of patient risk, as they are based on admission data and do not take into account the fluid nature of cardiogenic and/or non-cardiogenic shock, which may change during the entire period of the hospitalization. Hospital-wide platforms that integrate continuous EMR-based and/or EHR-based monitoring and prediction with a multidisciplinary alert system have the potential to favorably affect outcomes within this patient population.

By continuously risk-stratifying patients into appropriate shock stages (e.g., SCAI stages), improved efficiency in earlier identification of high-risk patients that may allow earlier intervention, including early TMS, can be achieved. This can allow for reduction in ICU LOS, intubation days, and use of renal replacement therapy as the early identification and alert provided by the SPE system can catch patients earlier on the spectrum of cardiogenic shock prior to development of hemometabolic shock.

As described above, the systems and methods described in the present disclosure can provide a platform of prospectively integrating real-time, continuous patient monitoring to risk-stratify both cardiogenic and non-cardiogenic shock patients into stages in conjunction with an integrated multidisciplinary alert and communication system as part of a comprehensive care pathway. Referring now to FIG. 7, an example workflow is illustrated for a multi-use pathway, in which the disclosed systems and methods identify the type of shock (e.g., cardiogenic shock, non-cardiogenic shock, mixed shock) and implement staging, paging, and/or engaging based on the determined shock type. As illustrated, the workflow includes receiving patient health data for shock assessment. The systems and methods can then generate shock stage classification data as described above to classify and/or risk stratify the type of shock.

When it is determined that cardiogenic shock is likely, intermediate actions can be instituted, such as recursively monitoring the patient's pulse pressure variation (PPV) or other physiological measurements. Additionally or alternatively, the continuously incoming patient health data (e.g., PPV data or other patient health data) can be input to the machine learning model to update or revise the shock stage classification data in real-time. As described above, paging and engaging can also be implemented, such as by generating a dedicated order set and/or pathway specific order set for the cardiogenic shock.

When it is determined that non-cardiogenic shock is likely, shock stage classification data that include differential scored data may be output to a user. If, based on the differential scored data the likelihood of cardiogenic shock outweighs the likelihood of non-cardiogenic shock, then the above-mentioned processing can be implemented (i.e., recursive monitoring of PPV or other physiological measurements, updating of the shock stage classification data in real-time). Otherwise, a dedicated order set and/or pathway specific order set may be generated for the non-cardiogenic shock type. Additionally or alternatively, as further patient health data are acquired they can be input to the machine learning model to update or revise the shock stage classification data in real-time.

FIG. 7 illustrates an example of a system 700 for generating shock stage scores for a patient in real-time based on patient health data, staging and/or risk stratifying a patient based on the shock stage scores, generating an alert and/or order set based on the staging and/or risk stratification for the patient, and communicating the alert and/or order set to a clinician, in accordance with some embodiments of the systems and methods described in the present disclosure. As shown in FIG. 7, a computing device 750 can receive one or more types of data (e.g., electronic medical record data, other types of patient health data) from data source 702. In some embodiments, computing device 750 can execute at least a portion of a stage, page, and engage system 704 to implement the staging module(s), paging module(s), and engagement module(s) described in the present disclosure, based on data received from the data source 702.

Additionally or alternatively, in some embodiments, the computing device 750 can communicate information about data received from the data source 702 to a server 752 over a communication network 754, which can execute at least a portion of the stage, page, and engage system 704. In such embodiments, the server 752 can return information to the computing device 750 (and/or any other suitable computing device) indicative of an output of the stage, page, and engage system 704.

In some embodiments, computing device 750 and/or server 752 can be any suitable computing device or combination of devices, such as a desktop computer, a laptop computer, a smartphone, a tablet computer, a wearable computer, a server computer, a virtual machine being executed by a physical computing device, and so on. The computing device 750 and/or server 752 can also reconstruct images from the data.

In some embodiments, data source 702 can be any suitable source of data (e.g., EMR data, EHR data, other patient health data), such as an EMR and/or EHR system or data store, another computing device (e.g., a server storing EMR, EHR data, or other patient health data), and so on. In some embodiments, data source 702 can be local to computing device 750. For example, data source 702 can be incorporated with computing device 750 (e.g., computing device 750 can be configured as part of a device for measuring, recording, estimating, acquiring, or otherwise collecting or storing data). As another example, data source 702 can be connected to computing device 750 by a cable, a direct wireless link, and so on. Additionally or alternatively, in some embodiments, data source 702 can be located locally and/or remotely from computing device 750, and can communicate data to computing device 750 (and/or server 752) via a communication network (e.g., communication network 754).

In some embodiments, communication network 754 can be any suitable communication network or combination of communication networks. For example, communication network 754 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 754 can be a local area network, a wide area network, a public network (e.g., the Internet), a private or semi-private network (e.g., a corporate or university intranet), any other suitable type of network, or any suitable combination of networks. Communications links shown in FIG. 7 can each be any suitable communications link or combination of communications links, such as wired links, fiber optic links, Wi-Fi links, Bluetooth links, cellular links, and so on.

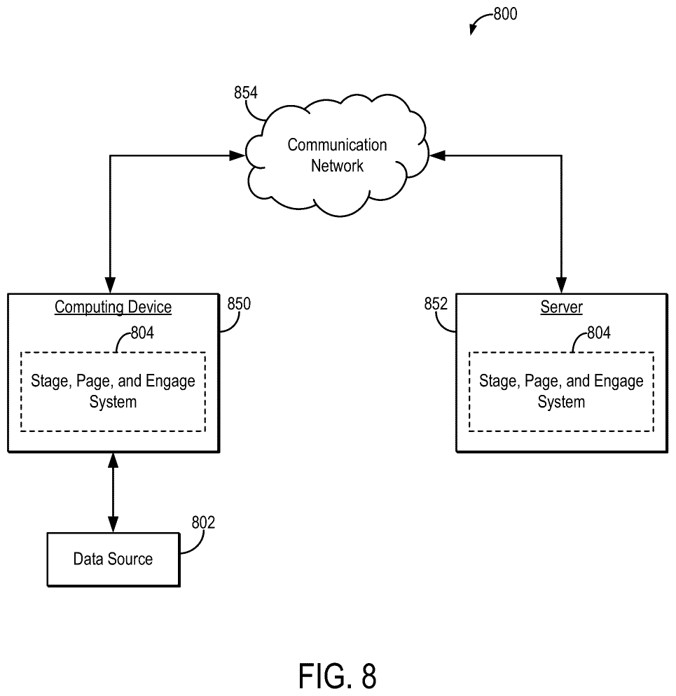

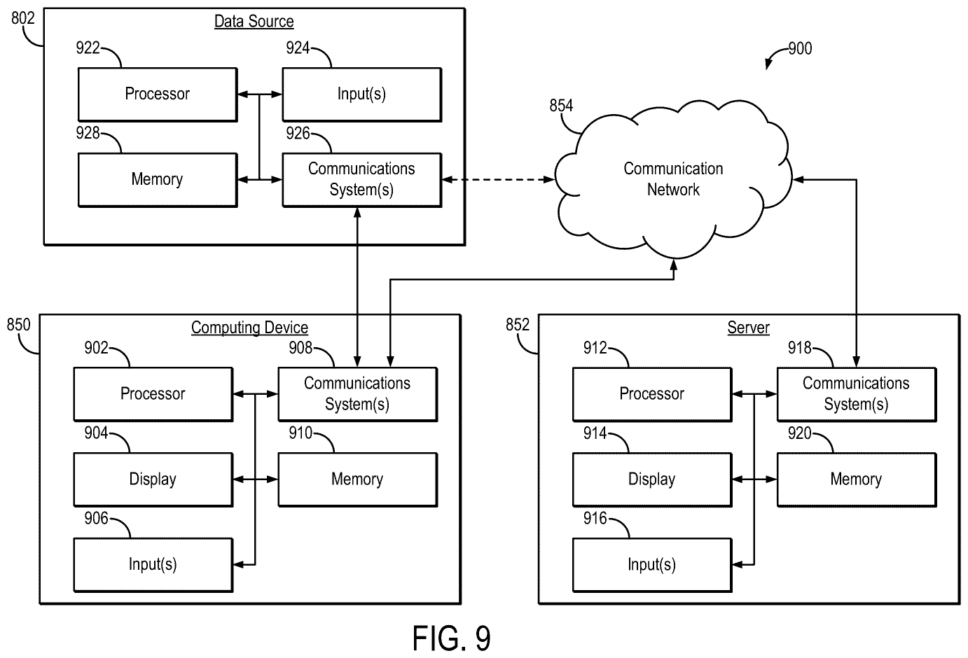

Referring now to FIG. 8, an example of hardware 800 that can be used to implement data source 702, computing device 750, and server 752 in accordance with some embodiments of the systems and methods described in the present disclosure is shown.

As shown in FIG. 8, in some embodiments, computing device 750 can include a processor 802, a display 804, one or more inputs 806, one or more communication systems 808, and/or memory 810. In some embodiments, processor 802 can be any suitable hardware processor or combination of processors, such as a central processing unit (“CPU”), a graphics processing unit (“GPU”), and so on. In some embodiments, display 804 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 806 can include any suitable input devices and/or sensors that can be used to receive user input, such as a keyboard, a mouse, a touchscreen, a microphone, and so on.

In some embodiments, communications systems 808 can include any suitable hardware, firmware, and/or software for communicating information over communication network 754 and/or any other suitable communication networks. For example, communications systems 808 can include one or more transceivers, one or more communication chips and/or chip sets, and so on. In a more particular example, communications systems 808 can include hardware, firmware, and/or software that can be used to establish a Wi-Fi connection, a Bluetooth connection, a cellular connection, an Ethernet connection, and so on.

In some embodiments, memory 810 can include any suitable storage device or devices that can be used to store instructions, values, data, or the like, that can be used, for example, by processor 802 to present content using display 804, to communicate with server 752 via communications system(s) 808, and so on. Memory 810 can include any suitable volatile memory, non-volatile memory, storage, or any suitable combination thereof. For example, memory 810 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 810 can have encoded thereon, or otherwise stored therein, a computer program for controlling operation of computing device 750. In such embodiments, processor 802 can execute at least a portion of the computer program to present content (e.g., images, user interfaces, graphics, tables), receive content from server 752, transmit information to server 752, and so on. For example, the processor 802 and the memory 810 can be configured to perform the methods described herein (e.g., the method of FIG. 6).

In some embodiments, server 752 can include a processor 812, a display 814, one or more inputs 816, one or more communications systems 818, and/or memory 820. In some embodiments, processor 812 can be any suitable hardware processor or combination of processors, such as a CPU, a GPU, and so on. In some embodiments, display 814 can include any suitable display devices, such as an LCD screen, LED display, OLED display, electrophoretic display, a computer monitor, a touchscreen, a television, and so on. In some embodiments, inputs 816 can include any suitable input devices and/or sensors that can be used to receive user input, such as a keyboard, a mouse, a touchscreen, a microphone, and so on.

In some embodiments, communications systems 818 can include any suitable hardware, firmware, and/or software for communicating information over communication network 754 and/or any other suitable communication networks. For example, communications systems 818 can include one or more transceivers, one or more communication chips and/or chip sets, and so on. In a more particular example, communications systems 818 can include hardware, firmware, and/or software that can be used to establish a Wi-Fi connection, a Bluetooth connection, a cellular connection, an Ethernet connection, and so on.

In some embodiments, memory 820 can include any suitable storage device or devices that can be used to store instructions, values, data, or the like, that can be used, for example, by processor 812 to present content using display 814, to communicate with one or more computing devices 750, and so on. Memory 820 can include any suitable volatile memory, non-volatile memory, storage, or any suitable combination thereof. For example, memory 820 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 820 can have encoded thereon a server program for controlling operation of server 752. In such embodiments, processor 812 can execute at least a portion of the server program to transmit information and/or content (e.g., data, images, a user interface) to one or more computing devices 750, receive information and/or content from one or more computing devices 750, receive instructions from one or more devices (e.g., a personal computer, a laptop computer, a tablet computer, a smartphone), and so on.

In some embodiments, the server 752 is configured to perform the methods described in the present disclosure. For example, the processor 812 and memory 820 can be configured to perform the methods described herein (e.g., the method of FIG. 6).

In some embodiments, data source 702 can include a processor 822, one or more inputs 824, one or more communications systems 826, and/or memory 828. In some embodiments, processor 822 can be any suitable hardware processor or combination of processors, such as a CPU, a GPU, and so on. In some embodiments, the one or more inputs 824 are generally configured to continuously record and/or retrieve patient health data in real-time, and can include various medical devices or sensors configured to measure a patient health variable (e.g., physiological sensors), and/or can include inputs for receiving patient health data posted to the patient's EMR and/or EHR. Additionally or alternatively, in some embodiments, the one or more inputs 824 can include any suitable hardware, firmware, and/or software for coupling to and/or controlling operations of a medical device, sensor, measurement system patient health data store, or the like. In some embodiments, one or more portions of the input(s) 824 can be removable and/or replaceable.

Note that, although not shown, data source 702 can include any suitable inputs and/or outputs. For example, data source 702 can include input devices and/or sensors that can be used to receive user input, such as a keyboard, a mouse, a touchscreen, a microphone, a trackpad, a trackball, and so on. As another example, data source 702 can include any suitable display devices, such as an LCD screen, an LED display, an OLED display, an electrophoretic display, a computer monitor, a touchscreen, a television, etc., one or more speakers, and so on.

In some embodiments, communications systems 826 can include any suitable hardware, firmware, and/or software for communicating information to computing device 750 (and, in some embodiments, over communication network 754 and/or any other suitable communication networks). For example, communications systems 826 can include one or more transceivers, one or more communication chips and/or chip sets, and so on. In a more particular example, communications systems 826 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 828 can include any suitable storage device or devices that can be used to store instructions, values, data, or the like, that can be used, for example, by processor 822 to control the one or more inputs 824, and/or receive data from the one or more inputs 824; to generate images from data; present content (e.g., data, images, a user interface) using a display; communicate with one or more computing devices 750; and so on. Memory 828 can include any suitable volatile memory, non-volatile memory, storage, or any suitable combination thereof. For example, memory 828 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 828 can have encoded thereon, or otherwise stored therein, a program for controlling operation of data source 702. In such embodiments, processor 822 can execute at least a portion of the program to generate images, transmit information and/or content (e.g., data, images, a user interface) to one or more computing devices 750, receive information and/or content from one or more computing devices 750, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for generating an order set for a patient based on a shock stage, the method comprising:
(a) receiving patient health data with a computer system, wherein the patient health data are associated with a patient and are continuously received in a real-time manner;
(b) accessing a staging algorithm with the computer system, the staging algorithm being configured to generate shock stage classification data from patient health data;
(c) inputting the patient health data to the staging algorithm using the computer system as the patient health data are continuously received by the computer system in real-time, generating an output as shock stage classification data for the patient;
(d) generating, with the computer system, an alert when the shock stage classification data for the patient indicate a change in a shock stage;
(e) generating, with the computer system and in response to the alert, an order set based on the shock stage classification data for the patient; and
(f) storing the order set in an electronic medical record (EMR) for the patient using the computer system.

2. The method of claim 1, wherein the shock stage classification data indicate an SCAI shock stage.

3. The method of claim 1, wherein the shock stage classification data indicate a numerical shock score value.

4. The method of claim 3, wherein the shock score value comprises a cardiogenic shock score value.

5. The method of claim 4, wherein generating the shock stage classification data further comprises correlating the cardiogenic shock score value with an SCAI shock stage using the computer system.

6. The method of claim 4, wherein the cardiogenic shock score value is computed based on data in the patient health data associated with measures of hypotension, lactate, vasopressor use, renal function, temporary mechanical support, and cardiac arrest.

7. The method of claim 1, wherein the shock stage classification data indicate a risk stage for deterioration of the patient.

8. The method of claim 1, wherein the patient health data comprise EMR data for the patient.

9. The method of claim 1, wherein the alert comprises an electronic message generated by the computer system.

10. The method of claim 9, wherein generating the alert comprises sending a page to a clinician by transmitting the page from the computer system to a pager.

11. The method of claim 1, wherein the alert further comprises a tier alert associated with an SCAI stage indicated by the shock stage classification data.

12. The method of claim 11, wherein the order set is updated based on a tier indicated by the tier alert.

13. The method of claim 11, wherein generating the alert comprises sending the alert to multiple users in a multidisciplinary health care team based on the tier indicated by the tier alert.

14. The method of claim 13, wherein generating the alert comprises providing, via the computer system, an access to a virtual videoconference room for the multiple users based on the tier indicated by the tier alert.

15. The method of claim 1, further comprising generating a care path for the patient and displaying the care path for the patient to a clinician via the computer system, wherein the care path provides a visual depiction of escalation pathways and de-escalation pathways between different shock stages for the patient.

16. The method of claim 1, wherein the staging algorithm comprises a machine learning model trained on training data to generate shock stage classification data from patient health data.

17. The method of claim 16, wherein the machine learning model is a supervised learning model.

18. The method of claim 1, wherein the shock stage comprises a cardiogenic shock stage.

19. The method of claim 1, wherein the shock stage comprises a non-cardiogenic shock stage.

20. The method of claim 19, wherein the non-cardiogenic shock stage comprises one of a disruptive shock stage, a hypovolemic shock stage, or an obstructive shock stage.

21. The method of claim 20, wherein the disruptive shock stage comprises a septic shock stage.

22. A non-transitory computer-readable media having stored thereon instructions that when executed by a processor cause the processor to perform a method comprising:
retrieving patient health data from a data storage in real-time, wherein the patient health data are associated with a patient;
accessing a machine learning model trained on training data to generate shock stage classification data from patient health data;
generating shock stage classification data for the patient in real-time by inputting the patient health data to the machine learning model as the patient health data are continuously retrieved from the data storage; and
storing the shock stage classification data using the processor.

23. The non-transitory computer-readable media of claim 22, wherein the method performed by the processor further comprises generating an alert when the shock stage classification data for the patient indicate a change in a shock stage.

24. The non-transitory computer-readable media of claim 23, wherein the method performed by the processor further comprises in response to the alert, generating an order set based on the shock stage classification data for the patient; and
storing the order set in an electronic medical record (EMR) for the patient.

25. A system for shock staging, comprising:
a staging module to generate shock stage classification data by:
receiving patient health data in real-time;
applying the patient health data to a staging model that classifies the patient health data as including features that are correlated with a particular shock stage classification;
a paging module to generate an alert in response to the shock stage classification data indicating a change in a shock stage; and
an engagement module to generate an updated order set for the patient in response to the shock stage classification data indicating the change in the shock stage.
